(19) United States
(12) Patent Application Publication
(10) Pub. No.: US 2005/0084415 A1
(43) Pub. Date: Apr. 21, 2005

(54) High capacity flash vapor generation systems

(75) Inventors: Iain F. McVey, Lakewood, OH (US); Francis J. Zelina, Lake City, PA (US); Aaron L. Hill, Erie, PA (US); Peter A. Burke, Concord, OH (US); Thaddeus Mielnik, Concord, OH (US); Matthew C. Lawes, Shaker Heights, OH (US); Gerald E. McDonnell, Basingstoke (GB); Kevin O. Williams, Solon, OH (US)

Correspondence Address: Thomas E. Kocovsky, Esq.;FAY, SHARPE, FAGAN, MINNICH & McKEE, LLP, Seventh Floor, 1100 Superior Avenue, Cleveland, OH, 44114-2518, US

(73) Assignee: STERIS, INC.

(21) Appl. No.: 10/940,495

(22) Filed: Sep. 14, 2004

Related U.S. Application Data
(63) Continuation-in-part of application No. 10/047,317, filed on Jan. 14, 2002.
(60) Provisional application No. 60/269,659, filed on Feb. 16, 2001.
(60) Provisional application No. 60/269,549, filed on Feb. 16, 2001.

(51) Int. Cl. A61L 2/20

(52) U.S. Cl. 422/28; 422/298; 422/305

(57) ABSTRACT

A flash vaporizer (34) provides a constant flow of vaporized hydrogen peroxide or other antimicrobial compounds for rapidly sterilizing large enclosures (10), such as rooms or buildings. The vaporizer includes a heated block (50) which defines an interior bore or bores (70, 72, 74). The flowpath created by the bore or bores increases in cross sectional area as the hydrogen peroxide passes through the block to accommodate the increase in volume during the conversion from liquid to gas. The vapor is injected into dry air in a duct that circulates it to the large enclosure.

This application claims the priority of U.S. Provisional Patent Application Ser. No. 60/269,659, filed February 16, 2001, U.S. Provisional Application Ser. No. 60/269,549, filed Feb. 16, 2001, and is a continuation in part of U.S. patent application Ser. No. 10/047,317, filed Jan. 14, 2002.

### BACKGROUND OF THE INVENTION

The present invention relates to the sterilization arts. It finds particular application in conjunction with hydrogen peroxide vapor systems used in connection with the sterilization of rooms, buildings, large enclosures, and bottling, packaging, and other production lines and will be described with particular reference thereto. It should be appreciated, however, that the invention is also applicable to other chemical vaporization systems such as those employing other peroxy compounds or aldehydes, for example, peracetic acid or formaldehyde vaporization systems.

Microbial decontamination of rooms and buildings can be achieved using chlorine dioxide gas. However, chlorine dioxide is highly toxic and must be recovered from the microbial decontamination process. Recovery of toxic gases from dilution air, leaking air, and the degassing of gas absorptive materials in the decontaminated room or building is difficult and time consuming. Further, care must be taken and monitors placed to insure that the toxic gas does not escape into the surrounding areas.

Sterile enclosures and other clean rooms are used by hospitals and laboratories for conducting tests in a microorganism-free environment. Further, a variety of medical, pharmaceutical, dental, and food packaging items are sterilized prior to use or reuse, in various forms of enclosures. Processing equipment for pharmaceuticals and food, freeze driers, meat processing equipment typically housed or moveable into large enclosures, or even rooms are advantageously sterilized.

Vaporized hydrogen peroxide is a particularly useful sterilant for these purposes because it is effective at low temperatures. Vaporized hydrogen peroxide systems provide dry, rapid, low-temperature decontamination of enclosed areas that are contaminated with microorganisms, including spore-forming bacteria. Keeping the temperature of the enclosure near room temperature eliminates the potential for thermal degradation of associated equipment and items to be sterilized within the enclosure. In addition, hydrogen peroxide readily decomposes to water and oxygen, which, of course, are not harmful to the humans including technicians, people nearby, or people subsequently entering the treated space.

For optimally effective sterilization, the hydrogen peroxide is maintained in the vapor state. Sterilization efficiency is reduced by condensation. Several different methods have been developed for delivering a vapor phase sterilant to an enclosure or chamber for sterilizing the load (e.g., medical instruments) or interior thereof. In one option, the “deep vacuum” approach, a deep vacuum is used to pull liquid sterilant into a heated vaporizer. Once vaporized, the sterilant diffuses by its vapor pressure into an evacuated and sealed chamber. In another option, the “flow-through” approach, vaporized sterilant is vaporized in a flow of carrier gas, such as air, that serves to deliver the sterilant into, through, and out of the chamber, which may be at a slightly negative or positive pressure. A solution of about 35% hydrogen peroxide in water is injected into the vaporizer as fine droplets or mist through injection nozzles. The droplets fall on a flat heated surface which heats the droplets to form the vapor, without breaking it down to water and oxygen. A carrier gas is circulated over the heat transfer surface to absorb the peroxide vapor.

As the size of the enclosure increases, or the demand for hydrogen peroxide is increased, the efficiency of the vaporization system becomes more significant. The capacity of the vaporizer is limited in a number of ways. First, the vaporization process creates a pressure increase, reducing the flow of air through the vaporizer. This increases the sterilization time and effectively limits the size of the enclosure to one which is capable of sterilization within an acceptable time period. Second, to maintain sterilization efficiency, the pressure at which the vapor is generated is limited to that at which the hydrogen peroxide is stable in the vapor state.

One solution has been to increase the size of the vaporizer, the injection rate of hydrogen peroxide into the vaporizer, and the flow rate of carrier gas. However, the carrier gas tends to cool the heating surface, disrupting the vaporization process. Heating the heating surface to a higher temperature breaks down the peroxide.

Yet another solution is to use multiple vaporizers to feed a single enclosure. The vaporizers may each be controlled independently, to allow for variations in chamber characteristics. However, the use of multiple vaporizers adds to the cost of the system and requires careful monitoring to ensure that each vaporizer is performing with balanced efficiency.

Large enclosures, such as buildings tend to become contaminated with a wide variety of microbial contaminants, including bacteria, molds, fungi, yeasts, and the like. These microorganisms thrive in damp spaces, such as behind walls, in plaster, under kitchen counters, in communal bathing/showering facilities, and the like, and tend to be very difficult to eradicate. For example fungi are allergenic agents and are occasionally infectious in susceptible people. They pose problems in buildings where moisture control is poor or water intrusion is common. Fungi grow in moist environments and form dormant, resistant spores, which are disseminated in the air. These spores tend to contact surfaces favorable for spore germination and outgrowth.

Fungi are also responsible for some of the indoor air sicknesses which occur in buildings which rely heavily on recirculating the air through air conditioning systems. Indoor air quality is affected, for example, by toxigenic spores released by Stachybotrys chartarum (black mold), Memnoniella, and Chaetomium globosum, among other species. Such spores, even if killed by conventional techniques, such as autoclaving, tend to cause sickness through inhalation of toxins released from the surfaces of the spores.

Additionally, fungi result in considerable commercial losses in the agriculture industry due to spoilage of food products. Germinating fungal spores tend to cause considerable damage to grains, nuts, beans, and the like, such as wheat, corn, soybeans, rice, and the like. Contamination may occur before or after harvesting. The germinating spores generate a variety of mycotoxins which are harmful to humans and animals on consumption, and thus are subject to strict regulation by the US EPA. Examples of such toxins include aflatoxins, ochratoxins, fumonisins, atranones, trichothecins, deoxynivenols, ergot alkaloids, and related compounds. Currently, food processing and bottling lines are treated to destroy aflatoxins by exposure to ammonia vapors, which may have an undesirable effect on the taste of the food product.

The present invention provides a new and improved vaporization system and method which overcomes the above-referenced problems and others.

### SUMMARY OF THE INVENTION

In accordance with one aspect of the present invention, a hydrogen peroxide vaporization system is provided. The system includes a block having an internal bore or bores which create a fluid flowpath through the block. A solution of hydrogen peroxide in water is passed along the flowpath. Increases in volume of the sterilant as it changes from liquid to vapor are accommodated by a progressively increasing size of the flowpath.

In accordance with another aspect of the present invention, a method of hydrogen peroxide vaporization is provided.

In accordance with another aspect of the present invention, a method of decontaminating an enclosure is provided. The method includes providing a first carrier gas stream and a second carrier gas stream, the first stream having a lower flow rate than the second stream. The first stream is introduced to a passage having at least one bend. A flow of an aqueous solution of a peroxy compound is introduced into the passage upstream of the bend. The peroxy compound mixes with the first stream. Walls of the passage are heated to vaporize the aqueous solution. The vaporized aqueous solution and first carrier gas stream is mixed with the second carrier gas stream in a mixing zone downstream of the passage and transported to the enclosure.

One advantage of the present invention is that a high output of vaporized hydrogen peroxide is achieved.

Another advantage of the present invention is that the air flow and hydrogen peroxpideeinjection rates can be increased.

Another advantage resides in the ability to decontaminate larger volumes.

Another advantage of the present invention is that it enables peroxide concentration levels to be raised rapidly to sterilization levels, particularly when used with smaller enclosures, thereby reducing the conditioning time.

Still further advantages of the present invention will become apparent to those of ordinary skill in the art upon reading and understanding the following detailed description of the preferred embodiments.

### BRIEF DESCRIPTION OF THE DRAWINGS

The invention may take form in various components and arrangements of components, and in various steps and arrangements of steps. The drawings are only for purposes of illustrating a preferred embodiment and are not to be construed as limiting the invention.

FIG. 1 is a schematic view of a preferred embodiment of a hydrogen peroxide vaporization system in accordance with the present invention;

FIG. 2 is a side sectional view of one embodiment of a vaporizer;

FIG. 3 is a perspective view of the vaporizer of FIG. 2;

FIG. 4 is a perspective view of a second vaporizer embodiment;

FIG. 5 is a side sectional view of a third vaporizer embodiment;

FIG. 6 is a side sectional view of a fourth vaporizer embodiment;

FIG. 7 is a side sectional view of a fifth vaporizer embodiment;

FIG. 8 is a diagrammatic illustration of an alternate system embodiment;

FIG. 9 illustrates another alternative system embodiment; and

FIG. 10 illustrates a system for decontamination of a building.

### DETAILED DESCRIPTION OF THE PREFERRED EMBODIMENTS

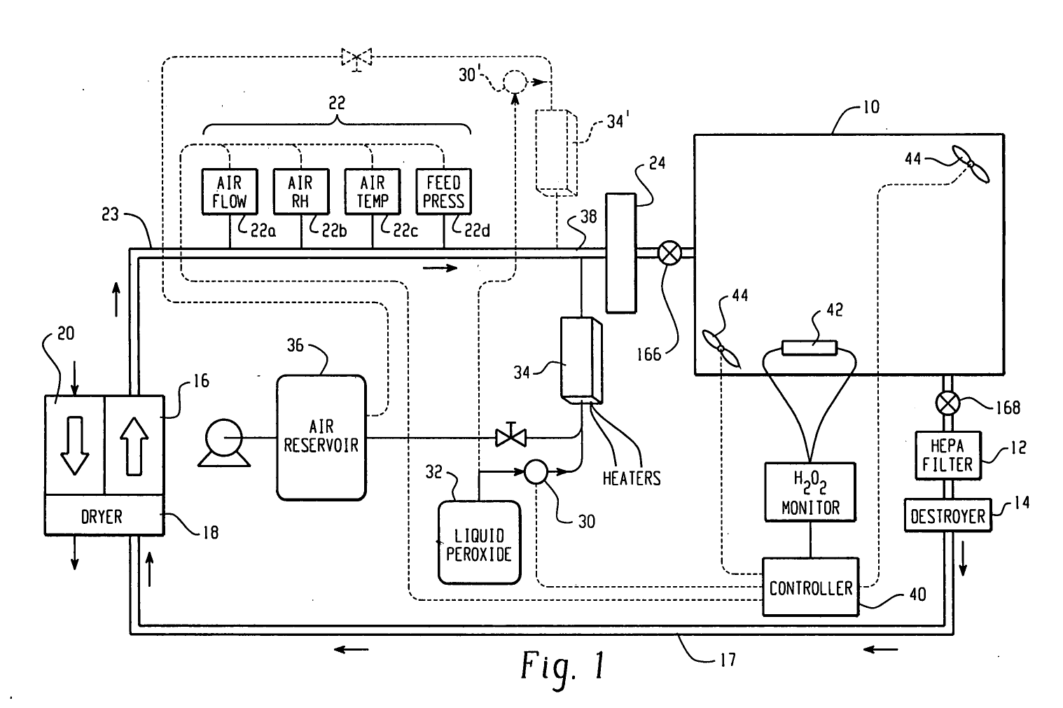

With reference to FIG. 1, a system for microbially decontaminating a room or other defined area with an antimicrobial vapor is shown. While the system is described with particular reference to hydrogen peroxide in vapor form, other antimicrobial vapors are also contemplated, such as vapors comprising peracetic acid or other peroxy compounds, aldehydes, such as formaldehyde vapors, and the like. Air from a large defined region, such as a room 10 with a volume on the order of 1,000-4,000 cubic meters is withdrawn through a contamination removing filter 12 and a peroxide destroying catalyst 14 by a blower 16, which is connected with the filter and destroyer by a duct or line 17. The blower optionally draws the air through a dryer, such as a desiccant wheel 18 which removes the water vapor. A second blower 20 blows heated air through a saturated portion of the desiccant wheel to remove and exhaust the absorbed moisture to the atmosphere. This heating process preferably heats the recirculated air from the ambient temperature of the room, typically about 20°-40° C. A series of air quality meters 22 monitor the dried air leaving the blower to determine its hydrogen peroxide vapor absorption capacity. The air is returned to the room 10 through a duct or line 23 and another microbe blocking filter 24, such as a HEPA filter. Optionally, the duct work includes all or a portion of a pre-existing HVAC system. Upon initially starting a decontamination process, the air is circulated through the dryer for a sufficient duration to bring the relative humidity in the room down to an acceptable level, preferably below 20% relative humidity. For sealed enclosures, pressure control within the enclosure may be appropriate. For rooms, pressure control is not essential and would be addressed on a case-by-case basis. In clean rooms and the like, where drawing potentially contaminated air into the room is to be avoided, the pressure in the room is maintained above ambient.

It will be appreciated that as an alternative to such a closed loop system, a flow through system may be employed in which the spent gas is vented or pumped from the room and any remaining hydrogen peroxide is destroyed before passing the vapor to atmosphere. In another embodiment, the air in the room is not dried prior to introducing hydrogen peroxide vapor.

Once the room has been brought to a sufficiently low relative humidity, an antimicrobial vapor is injected into the air. The antimicrobial vapor includes hydrogen peroxide vapor in the preferred embodiment, although other antimicrobial vapors or mixtures of antimicrobial vapors are also contemplated. More specifically, a means for introducing liquid hydrogen peroxide, such as an injection pump 30, pressurized container, gravity feed system, or the like, deposits hydrogen peroxide, preferably in the form of a liquid flow or spray, from a reservoir 32, such as a large drum, into a flash vaporizer 34. The liquid hydrogen peroxide includes a mixture of hydrogen peroxide in a diluent, such as water, preferably an aqueous mixture comprising about 30-40% by weight hydrogen peroxide in water. Optionally, a carrier gas, such as air, nitrogen, carbon dioxide, helium, argon, or a combination of carrier gases, is fed into the flash vaporizer concurrently with the hydrogen peroxide liquid to assist in propelling the peroxide vapor through the flash vaporizer and injecting it into the carrier gas flow. In a preferred embodiment, the carrier gas includes pressurized air from an air reservoir 36. The exact pressure varies with the production rate, the length and restrictiveness of passages in the flash vaporizer, and the like, and typically varies from 1.0-2.0 atmospheres absolute (1.013×10⁵-2.026×10⁵ Pascals absolute), i.e, about 0-1 atm. gauge (0-1.013×10⁵ Pascals gauge), more preferably, about 6-14×10³ Pa. An advantage of using such a carrier gas centers on the fact that the liquid hydrogen peroxide is unlikely to continuously impinge on the same point in the vaporizer. The more dispersed the liquid hydrogen peroxide is within the vaporizer, the more readily the peroxide will be vaporized. In addition, with a well dispersed hydrogen peroxide injection, the less likely that specific regions of the vaporizer will experience undue cooling thereby hindering the vaporization process.

The carrier gas tends to cool the vaporizer, reducing the rate at which the aqueous hydrogen peroxide solution is vaporized. Consequently, it is desirable to maintain the carrier gas at or slightly above a minimum flow rate needed to carry the vaporized hydrogen peroxide through the flash vaporizer 34 without significant degradation of the peroxide vapor, but at a flow rate which is low enough such that appreciable cooling of the vaporizer by the carrier gas does not occur. Accordingly, the flow rate of carrier gas through flash vaporizer 34 is preferably lower than the flow rate of carrier gas which does not pass through flash vaporizer 34. The majority of the carrier gas thus travels from the blower 16 through the duct 23 to a mixing zone 38 downstream of the vaporizer, where both the carrier gas stream and the vapor are combined prior to entering the enclosure. For example, the combined carrier gas streams may have a flow rate of about 20,000 liters/minute, while the carrier gas stream flowing through the flash vaporizer is 100 liters/min or less, more preferably, about 20 liters/min or less, most preferably, about 1-10 liters/min.

A controller 40 is connected with one or more peroxide concentration sensors 42 in the room. The controller controls fans 44 or other devices in the room 10 for adjusting the distribution of hydrogen peroxide vapor for better uniformity.

Based on the measured concentration in the room, the controller 40 controls the injection pump 30 and a feed rate of the air from the air reservoir 36 into flash vaporizer 34. The controller is further connected with air monitors 22 to maintain the injection rate below the saturation point of the circulated air. Preferably, the air quality monitors include an air flow monitor 22a for monitoring a rate of air flow, typically in the range of 20-40 cubic meters per minute. The monitors further include a relative humidity monitor 22b, an air temperature monitor 22c, and a pressure monitor 22d. When the air recirculation ducts are larger in diameter and have a higher air moving capacity, a second flash vaporizer 34′ and a second injection pump 30′ are connected with the liquid peroxide source 32 and with the air source 36. For larger enclosures, one or more additional air circulation lines with flash vaporizers are provided.

While described with particular reference to hydrogen peroxide, it will be appreciated that the system is also applicable to vaporization of other solutions and pure liquids, such as peracetic acid, other peroxy compounds, and the like.

The term “microbial decontamination” and similar terms, as used herein, encompass sterilization, disinfection, and lesser forms of antimicrobial treatment, such as sanitization. The term is also used to encompass the degradation or deactivation of other harmful biological species, particularly those capable of undergoing conformational changes, such as prions.

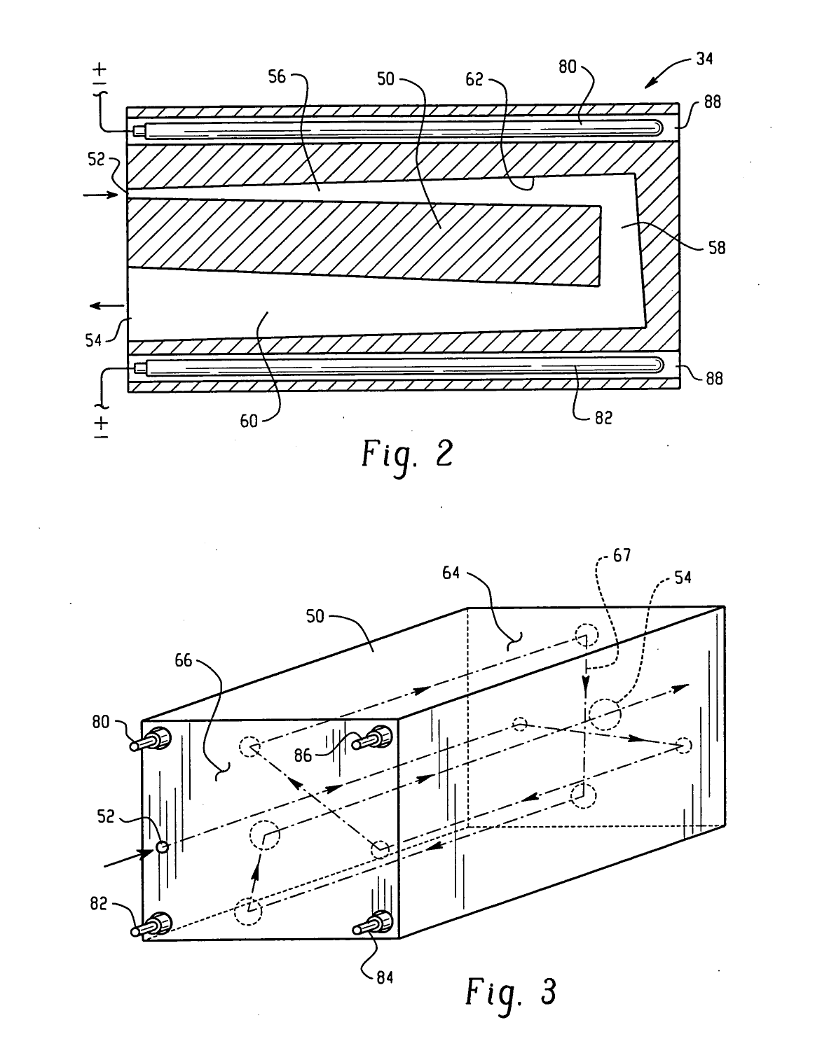

With reference also to FIG. 2, the flash vaporizer 34 includes a heated block 50, which is preferably formed from anodized aluminum, or other thermally conductive material resistant to hydrogen peroxide and with which the hydrogen peroxide is compatible, i.e., that does not degrade the hydrogen peroxide. A fluid pathway is defined by a one or series of bores, formed in the block extending from an inlet 52, connected with the supply line, to an outlet 54. In one embodiment, the series of bores 56, 58, 60 progressively increases in internal diameter from the inlet 52 to the outlet 54, thus creating an increasing area of contact and internal volume per unit length. The liquid hydrogen peroxide contacts the walls 62 of the bores and is vaporized. The increasing volume of the vapor/liquid mixture passing through the bore is accommodated by the increasing diameter of the bores.

In each of the embodiments, the bore may make several turns within the block. For example, starting at the bore inlet 52, the bore makes a U-turn adjacent an outlet end 64 of the block, returns to an inlet end 66 of the block, and makes two more such turns before reaching the outlet 54. Preferably, the turns are formed by sharp, “L-shaped” rather than rounded turns. For example, as shown in FIG. 3, each turn includes two approximately 90° corners and an end wall 67, which turn the bore through approximately 180°. Having generally sharp, rather than rounded corners encourages the flowing liquid/vapor mixture to hit the walls, thereby improving the rate of vaporization.

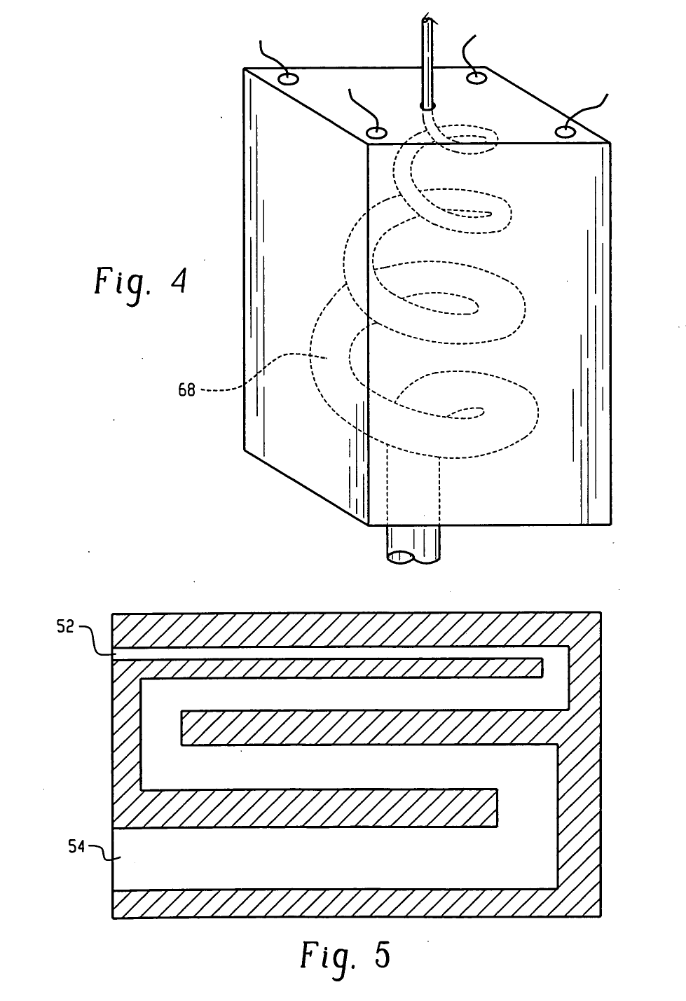

Other arrangements are contemplated, such as a spiral bore 68, as shown in FIG. 4. At each turn, inertia tends to propel fine, suspended droplets into the walls resulting in the vaporization of the droplets. In this manner, any fine droplets of mist or fog are turned to vapor. Preferably, at least two substantially 180° turns are provided in the flowpath to assure this increased contact.

The increasing diameter may be provided by progressively increasing the diameter of each segment of the bore, as shown in FIG. 2. Alternatively, longitudinal portions of the bore can each be of a single, successively larger diameter, as shown in FIG. 5. Other arrangements for progressively increasing the bore diameter are also contemplated. For example, baffles or fins may be provided adjacent the inlet to reduce the available flow space while increasing heated surface area.

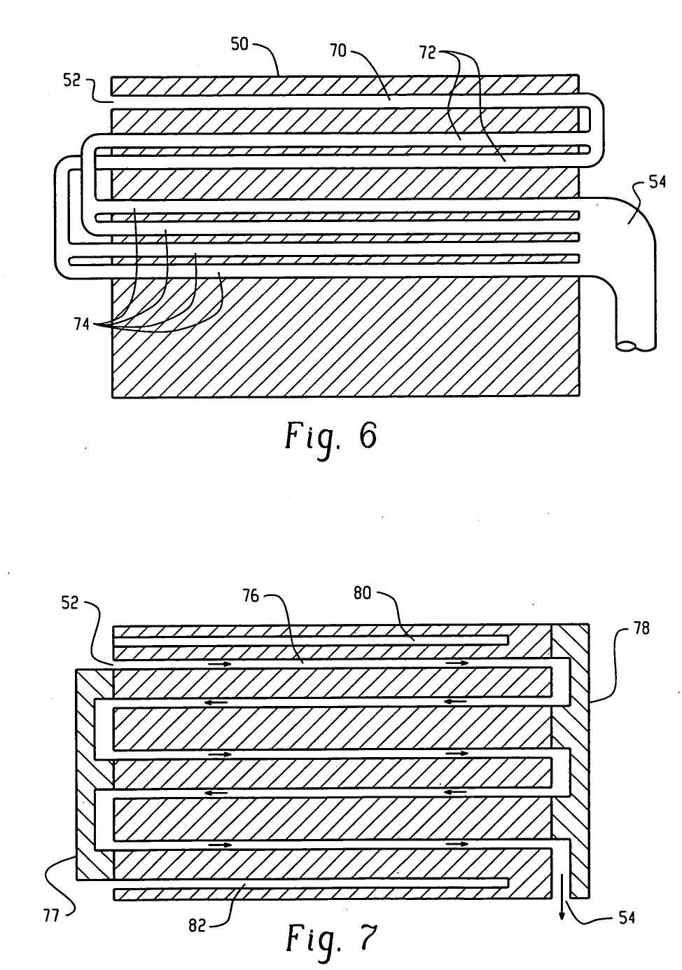

In the embodiment of FIG. 6, the number of bore portions increases with each pass through the block. For example, a single longitudinal bore 70 defines the first pass, two or more bore portions 72 define the second pass. Each of the second bores is preferably connected with more bores 74 for a third pass, and so forth. In this way, as for the earlier embodiments, the, cross sectional area of the fluid pathway created by the bores increases as the hydrogen peroxide travels from the inlet to the outlet (in this case, a plurality of outlets).

In an alternative embodiment, shown in FIG. 7, a bore 76 comprising one or more bore portions of uniform cross sectional area is provided, such that the entire bore or majority of the bore is of uniform cross sectional area. It is also contemplated that, for ease of manufacture, longitudinal bore portions may extend through the block, for example by drilling right through the block. The lateral portions are defined outside the block, by molded aluminum end pieces 77, 78, connecting tubing, or the like. The end pieces or connecting tubing are maintained at the temperature of the block and may be surrounded with a heating element, such as a heating tape with insulation, or the like.

With reference once more to FIGS. 2 and 3, block 50 is heated to a suitable temperature for vaporizing the liquid hydrogen peroxide. For example, heating elements 80, 82, 84, 86 are received in bores or passageways 88, preferably drilled longitudinally through the block adjacent the corners of the block. Suitable heating elements include electric resistance cartridge heaters. Such heaters are particularly appropriate for use as the heating element as they are commonly elongated and thin so that each heating element can be inserted into a heater bore and extend substantially from one end of the bore to the other. Alternatively, steam or another heated fluid is passed into heater bores to heat the block. The block is maintained by the heaters at a temperature below that at which significant dissociation of the hydrogen peroxide occurs.

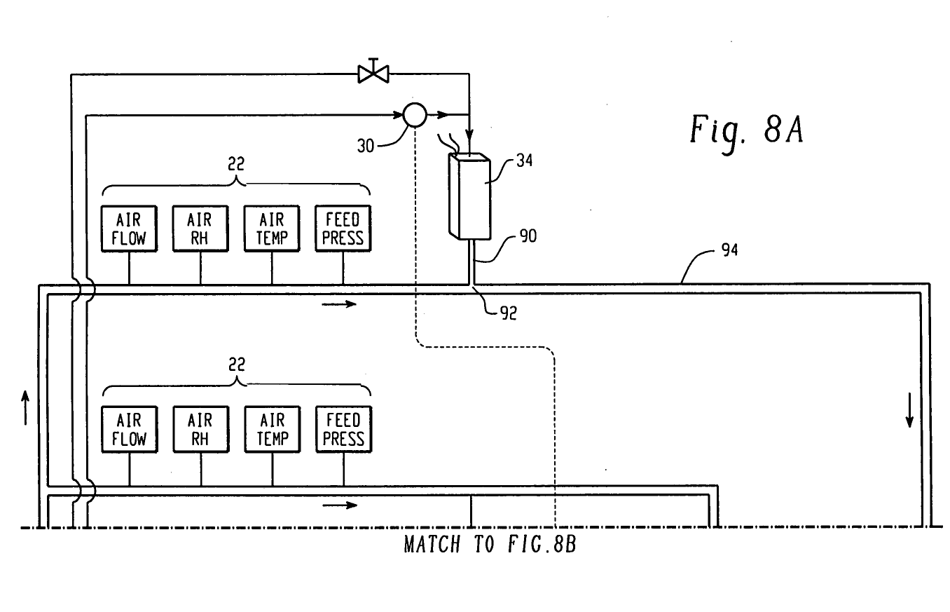

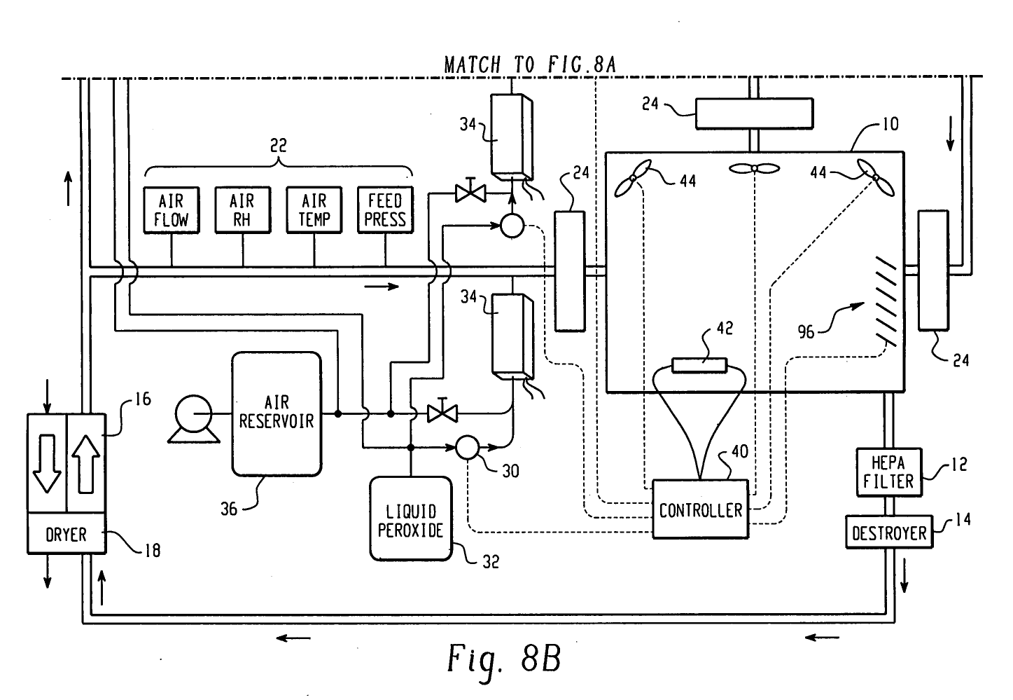

The liquid hydrogen peroxide vaporizes as it contacts the wall of the bore and is progressively converted from a liquid, spray, or mist to a vapor. The increasing pressure which would normally result from this conversion is substantially eliminated by the increase in size of the bore and/or by an increase in flow velocity such that the flow through the bore is maintained. At the end of the series of passes through the block, the hydrogen peroxide is preferably entirely in vapor form at a temperature and pressure which maintain the vapor below the dew point, such that condensation of the vapor does not occur. The hydrogen peroxide vapor is then entrained in a flow of a carrier gas. Specifically, as shown in FIG. 8, the vapor travels along a line 90 to an injection port 92, or other suitable injection device, which injects the vapor into a carrier gas line 94 at a mixing zone. The injection port 92 is defined at the edge of the duct 94 with a minimal extension into the air flow to minimize air flow cooling of the injection port which could lead to condensation. The hydrogen peroxide vapor has sufficient velocity to be impelled substantially across the duct as the vapor is mixed into the flowing air. When multiple flash vaporizers are used, the injection ports may be located across from each other and offset from each other to create swirling turbulence, up/downstream from each other, or the like.

With continuing reference to FIG. 8, in another embodiment, air from blower 16 and dryer 18 is divided among a plurality of supply lines. Each line is equipped with a series of monitors 22, a flash vaporizer 34, and a HEPA filter 24 as described above. Each of the lines injects peroxide vapor into a different region of the room or building 10. Based on concentration readings sensed by the sensors 42, the controller 40 causes fans 44 or baffles 96 to channel more or less air flow through some of the returns relative to others. Corresponding adjustments are made to the rate of vapor generation and injection into each return.

In order to achieve a desired level of disinfection or sterilization, it is important for the hydrogen peroxide vapor to contact all potentially contaminated surfaces in the room. The surfaces may include the walls, floor, and ceiling of the room as well as various surfaces of shelving, equipment, stored materials, and the like inside of the room. Fans 44 are positioned to urge the hydrogen peroxide vapor entering the room to flow against all surfaces. Particular attention is paid to occluded and difficult to reach surfaces. Fans or baffles are preferably positioned to urge the peroxide vapor into corners, through narrow gaps, under shelves, around complex objects, into narrow fissures and crevices, and the like.

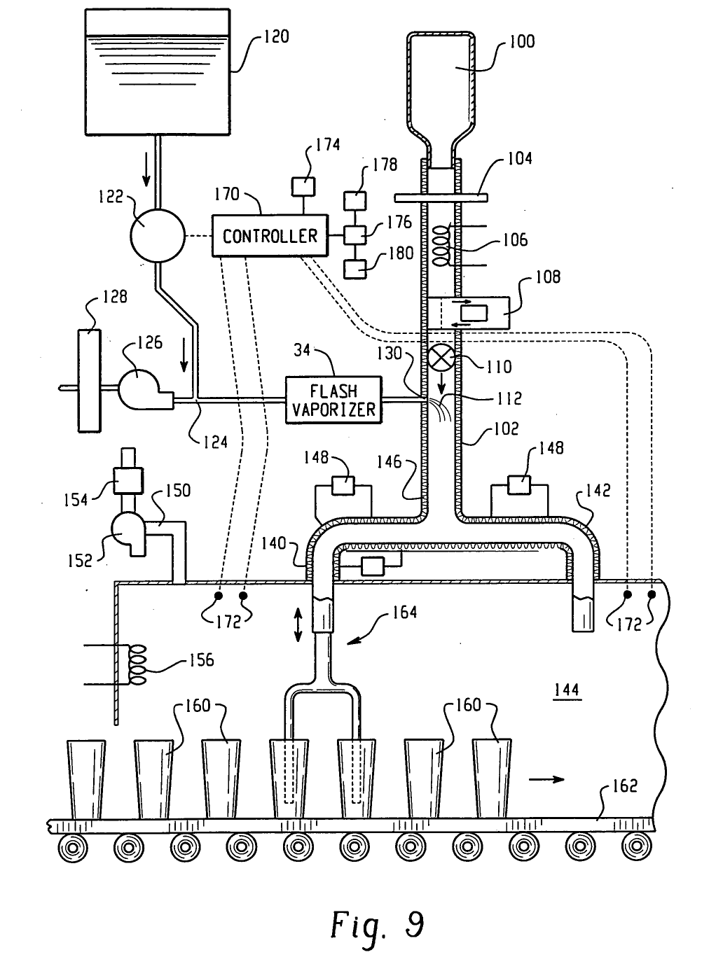

With reference again to FIG. 9, an open ended system is illustrated. A carrier gas is preferably air, although other gases which are unreactive towards hydrogen peroxide and the sterilized surfaces are also contemplated. A carrier gas generator 100 such as a pump or container of pressurized gas supplies the carrier gas to a duct 102. Microbe filters 104, such as HEPA filters, remove microbial and other particulate contaminants from the air. Preferably, a preheater 106 raises the temperature of the carrier gas. A dryer 108 preferably controls the humidity of the carrier gas. An adjustable baffle or gas flow regulator 110 controls the air flow rate to a peroxide absorption zone 112.

Liquid hydrogen peroxide (e.g., a water/hydrogen peroxide mixture) from a hydrogen peroxide supply 120 is pumped by a metering pump 122 to a mixing point 124 where it is mixed with filtered air from a blower 126 and a HEPA filter 128. The air and peroxide are injected into a flash vaporizer 34 as described above. The flash vaporizer injects hydrogen peroxide and water vapor through an injection port 130 into the absorption zone 112. Again, two or more vaporizers can be utilized to increase the rate of supply of peroxide gas to the absorption region.

Supply lines 140, 142 transport the mixture of carrier gas and vaporized hydrogen peroxide to a treatment site 144. To reduce the risk of condensation, the length of the supply lines 140, 142 is minimized. To reduce the risk of condensation further, insulation 146 and/or heaters 148 surround the supply lines 140, 142. Optionally, two or more supply lines connect each vaporizer to two or more regions of the enclosure 144. Optionally, the temperature of the carrier gas at the injection port may be increased to above the dew point of hydrogen peroxide.

A vent 150 permits controlled release of excess pressure in the enclosure. Optionally, a vacuum pump 152 evacuates the enclosure prior to hydrogen peroxide vapor introduction. Evacuation of the enclosure decreases the pressure and thus increases the diffusion rate of hydrogen peroxide therein. By reducing the pressure in the enclosure, one can minimize the need for baffles and/or fins at the point where the vaporized hydrogen peroxide is introduced into the enclosure. Alternatively, other types of pumps or blowers are used to help circulate and achieve a desired hydrogen peroxide concentration. Optionally, a catalyst 154 or the like breaks down any residual hydrogen peroxide in the vented gas. Optionally, a heater 156 raises the temperature of and within enclosure 144 prior to and during microbial decontamination. Raising the temperature in the enclosure or at least its surfaces also reduces the tendency for vapor to condense.

Sterilizable enclosures include microorganism-free work areas, freeze dryers, and pharmaceutical or food processing equipment. Whether high sterilization temperatures and/or evacuation of the enclosure during sterilization are feasible depends on the construction of the enclosure and the nature of its contents. For example, sterilizable work areas are, in some instances, constructed of non-rigid plastic materials which do not withstand high temperatures and large pressure gradients. Food processing equipment, in contrast, is often required to withstand high temperatures and pressures during processing operations and is more easily adapted to achieving more optimal sterilization conditions through evacuation and heating.

In FIG. 9, enclosure 144 is a portion of a packaging plant. Containers, such as bottles or cartons 160 are carried into the enclosure on a conveyor system 162. A reciprocating manifold 164 is connected with the each of the supply lines 140, 142 and sequentially raises and lowers a number of fill tubes or peroxide vapor injectors into the bottles or cartons as they pass or are indexed.

The hydrogen peroxide concentration in the solution is selected according to the desired vapor concentration. For example, the hydrogen peroxide concentration may be from 25-65% by weight aqueous hydrogen peroxide. In one embodiment, the hydrogen peroxide concentration is from about 30-35% by weight aqueous hydrogen peroxide. At this level, condensation of hydrogen peroxide is limited, while microbial decontamination is achieved in a short period of time.

In one embodiment, the hydrogen peroxide vapor is maintained at a concentration in the enclosure 144 until microbial decontamination is complete, and is continually replenished to maintain prescribed concentration levels. Optionally, the vacuum pump 152 draws out the hydrogen peroxide vapor from the enclosure following microbial decontamination. This reduces the time required for dissipation of the hydrogen peroxide, and returns the enclosure to useful activity more quickly. Alternatively or additionally, the enclosure is aerated, for example, by passing carrier gas alone through the enclosure, to remove any remaining hydrogen peroxide. In addition, a sensor may be employed to confirm that the enclosure has been aerated and that it may be returned to normal use.

Alternatively, once the hydrogen peroxide concentration of the enclosure has achieved a desired level, the vapor is held in the enclosure for a selected period of time sufficient to effect decontamination, without further additions of vapor to the enclosure or withdrawals of gas and/or vapor from the enclosure. For example, as shown in FIG. 1, valves 166, 168 in the vapor inlet and outlet lines leading to and from the enclosure are selectively closed once a selected vaporized hydrogen peroxide concentration is detected, and the hydrogen peroxide is held in the enclosure for a period of about one hour. For room-sized enclosures, in particular, it has been found that the hydrogen peroxide does not degrade or condense too rapidly in this time, such that microbial decontamination generally occurs throughout the holding period. The valves are then reopened and the remaining hydrogen peroxide is withdrawn. In a further embodiment, a series of two or more hold periods is used. In between each successive hold period, the hydrogen peroxide concentration is readjusted to the desired level.

In the illustrated embodiment, vaporizer 34 is preferably located in close proximity to the enclosure. Where more than one vaporizer is used, the rate of introduction of hydrogen peroxide by the individual vaporizers is adjustable so as to optimize hydrogen peroxide vapor distribution within the enclosure.

Differences in temperature and absorbency of materials within the enclosure, flow patterns in the enclosure, and enclosure shape are among the factors influencing the optimum rate of introduction. In the flow-through system of FIG. 9, the rate of throughput of containers or bottles through the enclosure also influences the optimum rate of peroxide introduction. Preferably, a control system 170 regulates the introduction of hydrogen peroxide to the flash vaporizer(s) 34 in accordance with detected conditions within the enclosure. A plurality of monitors 172 monitor conditions within the enclosure 144. The monitors include temperature sensors, humidity or vapor concentration sensors, air flow or turbulence sensors, pressure sensors, and the like. The control system includes a comparator 174 for comparing the monitored condition signals from the monitors with preselected ideal hydrogen peroxide vapor concentration and other conditions as indicated by reference signals. Preferably, the comparator determines a deviation of each monitored condition signal from the corresponding reference signal or a reference value. Preferably, a plurality of the conditions are sensed and multiple comparators are provided. A processor 176 addresses an algorithm implementing program or pre-programmed look up table 178 with each deviation signal (or combination of deviations of different conditions) to retrieve a corresponding adjustment for each flash vaporizer 34. Other circuits for converting larger deviations to larger adjustments and smaller deviations to smaller adjustments are also contemplated. Alternately, the error calculation can be made at very short intervals with constant magnitude increases or decreases when the monitored condition is below or above the reference points.

The adjustment values adjust the hydrogen peroxide metering pump 122 and the carrier gas regulator 110 to bring the monitored conditions to the reference values. For example, vapor injection rates are increased by vaporizers near regions with lower vapor concentration, higher temperatures, higher pressure, and the like. Vapor production rates are reduced in response to higher sensed vapor concentration, lower sensed temperatures, lower pressure, and the like. The processor, optionally, also controls the enclosure heater 156, circulation fans in the enclosure, the vacuum pump 152, or the like. Optionally, an operator input 180 enables the operator to adjust the reference signal in each region to cause higher or lower concentrations in selected regions.

Flash vaporizer 34 is capable of achieving a higher vapor output than conventional, drip-type vaporizers. For example, a heating block which supplies 1653 watts to the bores is able to vaporize 50 grams of hydrogen peroxide/minute (35% hydrogen peroxide, 65% water), since the heat of vaporization of the solution is 33.07 watt-min/gram. Obviously, as the heat supplied increases, correspondingly higher outputs can be achieved. Using one or more of such vaporizers, a high speed bottling line (e.g., about 1000 bottles/min) can be decontaminated.

One specific embodiment of the application is in the removal of microorganisms, particularly bacteria, fungi, and viruses, and the toxins associated with such microorganisms, from buildings, such as factories, hospitals, schools, research laboratories, communal bathing/showering facilities, and residential buildings. The hydrogen peroxide vapor treatment has been found to be effective against a variety of fungi and their spores, including Stachybotrys chartarum, Aspergillus niger, Chaetomium globosum, and Trichophyton mentagrophytes, which are responsible for a variety of cutaneous and respiratory illnesses, especially in people having compromised immune systems.

The hydrogen peroxide vapor treatment is also effective against a wide variety of man made or refined contaminants, such as chemical and biological warfare agents. Biological warfare agents include biological microorganisms employed to disable personnel, as well as pesticides, herbicides, and similar substances which can be employed to interfere with the growth of plants, insects, and other non-mammalian species. Dissemination of such agents is achieved with aerosol sprays, explosives, food and water contamination, mail systems, and the like. They are commonly dispersed in aerosol form, as fine particles are most effective as biological weapons. Included among these are viruses, such as equine encephalomyelitis, Ebola, and smallpox (Variola); bacteria, such as those which cause plague (Yersina pestis), anthrax (B anthracis), brucellosis (e.g., Brucella melitensis, Brucella suis, Brucella abortus, and Brucella canis), and tularemia (Francisella tularensis); cholera (Vibrio cholerae), and fungi, such as Fusarium, Myrotecium and coccidioidomycosis; as well as toxic products expressed by such microorganisms; for example, the botulism toxin expressed by the common Clostridium botulinium bacterium, and ricin, a plant protein toxin derived from the beans of the castor plant.

These microoganisms may have been refined, purified, or otherwise treated to increase their potency, such as in weapons grade anthrax. The hydrogen peroxide vapor reduces the activity of the microbial or chemical contaminant, either by killing a majority of the contaminant, as in the case of a microbial contaminant, or by converting the contaminant to a less harmful material, as in the case of a chemical contaminant.

Chemical warfare agents include poison gases and liquids, particularly those which are volatile, such as nerve gases, blistering agents (also known as vesicants), and other extremely harmful or toxic chemicals. They are commonly dispersed as gases, smoke, or aerosols. Missiles, rockets, spray tanks, landmines, and other large munitions are often employed. As used herein, the term “chemical warfare agent” is intended to include only those agents which are effective in relatively small dosages to substantially disable or kill mammals. The term “chemical warfare agent” is not intended to encompass incendiaries, such as napalm, or explosives, such as gunpowder, TNT, nuclear devices, and so forth.

Exemplary chemical warfare agents include choking agents, such as phosgene; blood agents, which act on the enzyme cytochrome oxidase, such as cyanogen chloride and hydrogen cyanide; incapacitating agents, such as 3-quinuclidinyl benzilate (“BZ”), which blocks the action of acetylcholine; vesicants, such as di(2-chloroethyl) sulfide (mustard gas or “HD”) and dichloro(2-chlorovinyl)arsine (commonly known as Lewisite); nerve agents, such as ethyl-N, N dimethyl phosphoramino cyanidate (commonly known as Tabun or agent GA), o-ethyl-S-(2-diisopropyl aminoethyl) methyl phosphono-thiolate (commonly known as agent VX), isopropyl methyl phosphonofluoridate (commonly known as Sarin or Agent GB), methylphosphonofluoridic acid 1,2,2-trimethylpropyl ester (commonly known as Soman or Agent GD). Sarin, for example, is an extremely active cholinesterase inhibitor with a lethal dose for man as low as 0.01 mg/kg body weight. Soman also has a lethal dose as low as 0.01 mg/kg body weight.

The term “chemical warfare agent” includes substantially pure chemical compounds, but the term also contemplates mixtures of agents in any proportions, as well as those agents in impure states. “Chemical warfare agents,” as used herein, also includes partially or completely degraded chemical warfare agents, e.g., gelled, polymerized, or otherwise partially or totally decomposed chemical warfare agents.

The hydrogen peroxide system is particularly effective at destroying these chemical and biological warfare agents and other harmful oxidizable species because it is capable of generating a large vapor output. Large rooms and other enclosures can be decontaminated with the vapor, as well as items placed in an enclosure, such as clothes, weapons, vehicles, other military equipment, and the like. For example, protective clothing and equipment exposed to such chemical and biological warfare agents can be decontaminated with the hydrogen peroxide vapor without the need for destruction by burning. In view of the potential for liberating harmful chemicals during incineration, such processes are preferably avoided.

In one embodiment, windows, doors and other openings to the environment are substantially sealed and the flash hydrogen peroxide vaporizer is connected with ductwork which supplies air throughout the building, such as an HVAC (heating, ventilating and air conditioning) system. The HVAC system carries the hydrogen peroxide throughout the building and may supply the stream of carrier gas (air) which mixes with the air and hydrogen peroxide vapor stream supplied by the vaporizer. The hydrogen peroxide vapor is flowed through the building for a sufficient time to destroy microorganisms present in the air (if an airborne contamination is detected), or within walls and other structural parts of the building, if more serious contamination is detected. Typically, an exposure time of about twenty to thirty minutes is sufficient to provide time for the vapor to penetrate into less accessible areas of the room or building and ensure destruction of the harmful microorganisms. After the decontamination phase is complete, air is circulated through the building to flush residual hydrogen peroxide from the building, preferably first passing the spent vapor through a catalytic converter to convert the hydrogen peroxide to water and oxygen. Windows and doors are optionally opened to speed the removal, particularly if the building is sufficiently far from other areas of human activity to ensure that hydrogen peroxide is rapidly dissipated through the air.

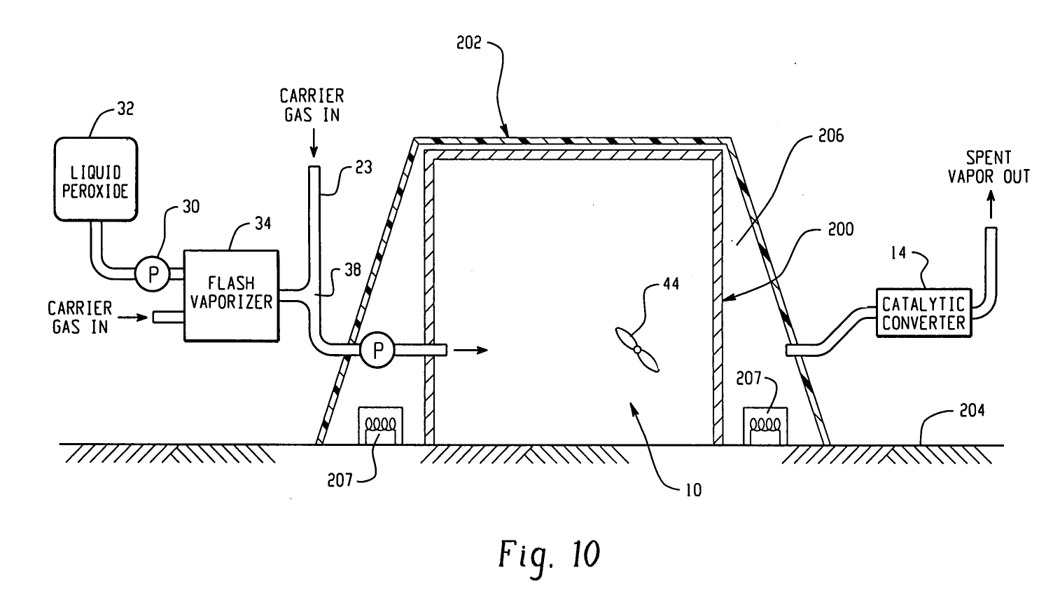

In another embodiment shown in FIG. 10, where like components are accorded the same numerals and new components are accorded new numerals, a building 200 or portion thereof to be microbially decontaminated is tented with a temporary enclosure 202, such as a plastic tent. The tent 202 may be formed by joining sheets of flexible plastic together to create a substantially airtight enclosure. The enclosure is staked or otherwise tied to the ground 204 around the building to provide an enclosed space 206 in which a hydrogen peroxide concentration can be maintained. The flash vaporizer 34 and carrier gas supply line 23 are fluidly connected with the enclosure 202, or directly with the building ventilation system, through an opening in the enclosure. Spent vapor is passed through a catalytic converter 14 upon leaving the enclosure. Heaters 207 may be provided within the enclosure to reduce the likelihood of condensation of the vapor. Heating in the building is preferably set at a level which minimizes condensation. By heating the building above ambient temperature, e.g., to a temperature of about 30° C. or above, higher levels of hydrogen peroxide can be sustained and faster decontamination achieved.

Another application is in the treatment of food storage facilities, or their contents, such as grain silos, barns, and the like, using analogous method to those described for residential or public buildings. In another embodiment, food processing lines or beverage bottling lines are treated with vapor from the vaporizer to destroy microorganisms or the toxins they generate.

For example, mycotbxins generated by germinating spores are destroyed or otherwise, rendered non-toxic by treatment with the vapor. Examples of such toxins include aflatoxins, ochratoxins, fumonisins, atranones, trichothecins, deoxynivenols, ergot alkaloids, and related compounds. Food processing and bottling lines are readily treated to destroy aflatoxins and other mycotoxins by exposure to the hydrogen peroxide vapor, which does not have the undesirable effect on the taste of the food product that conventional treatments do.

One advantage of using hydrogen peroxide in all of the applications discussed herein is that it is not necessary to make sure that the area to be treated is dry before introducing the hydrogen peroxide vapor. The temperature and humidity of the region to be treated are determined and the concentration of hydrogen peroxide in the vapor is controlled to keep it below the condensation point.

Hydrogen peroxide vapor has been found to be effective at both high and low humidity levels. Thus, it is not necessary to dry the air initially present in the region or to dry the carrier gas. In a closed loop system, for example, the spent vapor can be recirculated through the vaporizer without drying the air. If appropriate, the concentration of the hydrogen peroxide can be maintained by selectively increasing or decreasing the amount of liquid hydrogen peroxide entering the vaporizer.

Tests show the effectiveness of hydrogen peroxide for destruction of a wide variety of microorganisms. The following Examples, which are not intended to limit the invention, show the effectiveness of vapor hydrogen peroxide for treatment of fungi.

### EXAMPLES

### Example 1

The microbiocidal effectiveness of vaporized hydrogen peroxide against several strains of fungi considered to be of concern to human health and building contamination is evaluated. Five fungi strains, Stachybotrys chartarum ATCC 34915 (European strain), Stachybotrys chartarum ATCC 201212 (USA strain), Chaetomium globosum ATCC 58948, Aspergillus niger ATCC 6275, and Trichophyton mentagrophytes ATCC 18748, are exposed to hydrogen peroxide vapor as dried (viable) fungal spore preparations on stainless steel carriers for 0.5, 1, 3, 5, and 7 minutes, and then evaluated for test organism recovery after exposure to the vapor.

The studies are carried out in a small enclosure using a VHP® 1000 sterilizer available from STERIS Corp., Mentor, Ohio. The sterilizer is a compact, mobile unit which generates, delivers, controls and removes hydrogen peroxide vapor for an enclosed environment. The VHP® 1000 includes a microprocessor which continuously monitors, controls and documents the process parameters during each cycle.

Stainless steel test carriers (coupons) are cleaned and steam sterilized before use. For each test organism, plate cultures are prepared by transferring one colony from a stock culture slant and streaking it onto the surface of an agar plate (corn meal agar for S. chartarum and C. globosum, potato dextrose agar for A. niger, and Sabouraud (SAB) dextrose agar for T. mentagrophytes). Plate cultures are incubated at the appropriate temperatures for each test organism, as shown in TABLE 1. When spore formation of the test organism has occurred (determined visually and microscopically), the spores are harvested from the agar plate culture with 2.0 mL of sterile deionized (DI) water and gentle rubbing. The spores are pelleted by centrifugation (10,000 rpm setting, 3 minutes, ambient temperature). The supernatant is discarded and the pellet is resuspended in sterile water. A direct count of the initial spores is performed using a Petroff-Hausser counting chamber and phase contrast microscopy and centrifugation or dilution is used as needed to adjust the count to 1-4×10⁸ spores/mL.

TABLE 1

| Test Organism | Recovery Agar Media | Incubation Conditions |
|---|---|---|
| Chaetomium | Sabouraud dextrose agar | Ambient temperature, in |
| globosum ATCC | | the dark |
| 58948 | | |
| Aspergillus niger | Potato dextrose agar | 30 ± 1° C., in the dark |
| ATCC 6275 | | |
| Trichophyton | Sabouraud dextrose agar | 30 ± 1° C., in the dark |
| mentagrophytes | | |
| ATCC 18748 | | |
| Stachybotrys | Sabouraud dextrose agar | Ambient temperature, in |
| chartarum ATCC | | the dark |
| 34915 | | |
| Stachybotrys | Sabouraud dextrose agar | Ambient temperature, in |
| chartarum ATCC | | the dark |
| 201212 | | |

For each test organism, twenty sterile stainless steel test carriers are inoculated with 10 μL of the appropriate fungal spore suspension and air-dried at ambient temperature. Sample test carriers are evaluated to ensure that each test carrier provides an average viable inoculum of 1×10⁶CFU (colony forming units)/test carrier.

A 22 cubic feet flexible wall transfer isolator at ambient temperature is dehumidified using an air flow rate of 15 SCFM (standard cubic feet per minute) to an absolute humidity of 2.3 mg/L over a time of 20 minutes. In a conditioning phase, liquid hydrogen peroxide (35% hydrogen peroxide) is introduced at 2.5 g/minute into carrier gas at an air flow rate of 12 SCFM for twenty minutes and flowed through the isolation chamber. In a decontamination phase, the injection rate is 1.8 g/minute and air flow rate is maintained at 12 SCFM for at least thirty minutes. An aeration phase is carried out for 60 minutes at an air flow rate of 20 SCFM.

The isolation chamber is adapted with an access port (D-tube) that allows for the introduction and removal of test coupons during the decontamination phase of the cycle. The inoculated test carriers are suspended in the D-tube by hanging the carriers on wire hooks so that each test carrier hangs freely without contacting any other surfaces. Exposure times for each test organism are 0.5, 1, 3, 5, and 7 minutes. Three test carriers are evaluated at each exposure time for each test organism.

Immediately after exposure, each carrier is aseptically transferred to 10.0 mL of 0.01% catalase neutralizing solution (9.0 mL DI water and 1.0 mL of 0.1% catalase solution) and swirled to mix.

Viable test organisms from the test carriers after exposure to the vapor are extracted by sonication in the neutralization solution, dilution, and filtering through a 0.45 μm membrane (Nalgene™ sterile filter). The membranes are transferred to the appropriate recovery media for the test organism (TABLE 1). Then, 5 mL of trypticase soy broth is added to each of the empty, carrier-containing tubes to recover any fungal spores that may still be attached to the carrier. All plates are incubated at the appropriate conditions for each test organism for 6-8 days and 8 days incubation for tubes. Plate counts are used to calculate the average log reduction of each test organism. After the tube incubation period, each tube is recorded as growth (+) or no growth (−).

TABLE 2 shows the average initial (viable) test carrier population for each test organism and log reductions after exposure. The average initial test carrier populations ranged from 1.2-2.5×10⁶ CFU/carrier.

TABLE 2

| | | Log₁₀ | |
|---|---|---|---|
| | Average Initial | Reduction (Complete | |
| | Test Carrier | Kill) with VHP | |
| | Population (Fungal | Exposure | Log₁₀ |
| Test Organism | spores/carrier) | Time (Min) | Reduction |
| Chaetomium globosum | 1.3 × 10⁶ | 1 | 6.1 |
| ATCC 58948 | | | |
| Aspergillus niger ATCC | 2.3 × 10⁶ | 1 | 6.4 |
| 6275 | | | |
| Trichophyton | 1.2 × 10⁶ | 1 | 6.1 |
| mentagrophytes ATCC | | | |
| 18748 | | | |
| Stachybotrys chartarum | 2.5 × 10⁶ | 3 | 6.4 |
| ATCC 34915 | | | |
| Stachybotrys chartarum | 1.2 × 10⁶ | 5 | 6.1 |
| ATCC 201212 | | | |

As shown in TABLE 2, hydrogen peroxide vapor is found to be effective against all of the fungi tested, demonstrated by a 6-log reduction (less than 1 in 1,000,000 viable spores remaining, i.e., a “total kill”) of Chaetomium globosum, Aspergillus niger, and Trichophyton mentagrophytes within a 1-minute exposure to hydrogen peroxide vapor. For Stachybotrys chartarum, a 6-log reduction (total kill) at 3 and 5 min is achieved for strains 34915 and 201212, respectively. Since these five strains are representative of hard to kill fungi which pose hazards to humans and lead to building contamination, treatment of entire rooms or buildings with hydrogen peroxide vapor is expected to result in rapid destruction of these and other fungal strains.

The somewhat higher resistance of the two S. chartarum strains may be due in part to the larger spore size of the Stachybotrys strains as compared to the other fungi strains tested, leading to a denser packing on the test carrier. Additionally, the spores are coated with a slime layer that eventually dries over the surface of the spores. These two factors may result in a greater penetration challenge to the vapor, thereby extending the kill time of the two Stachybotrys strains.

The invention has been described with reference to the preferred embodiment. Obviously, modifications and alterations will occur to others upon reading and understanding the preceding detailed description. It is intended that the invention be construed as including all such modifications and alterations insofar as they come within the scope of the appended claims or the equivalents thereof.

1. A vapor decontamination system for decontaminating a defined region, the system comprising:
at least a first duct along which a carrier gas is passed to the defined region;
a flash vaporizer for vaporizing a liquid which includes an antimicrobial compound into vapor, an outlet of the flash vaporizer being connected to the duct for supplying the vapor into the duct for absorption into the carrier gas passing through the duct at a mixing zone;
a means for introducing the liquid from a source to the flash vaporizer.

2. The system as set forth in claim 1, wherein the antimicrobial compound includes hydrogen peroxide and the flash vaporizer includes:
a metal block;
at least one heater for heating and maintaining the metal block at or above a vaporization temperature of hydrogen peroxide and below a hydrogen peroxide disassociation temperature; and
a passage extending through the block from an inlet to the outlet.

3. The system as set forth in claim 2, wherein the passage expands in cross section between the inlet and the outlet.

4. The system as set forth in claim 3, wherein the passage turns at least 180° between the inlet and the outlet.

5. The system as set forth in claim 4, wherein the passage includes at least two turns of approximately 90° and a wall therebetween, such that the liquid in the passage strikes the wall, thereby increasing a vaporization rate of 5 the liquid antimicrobial compound.

6. The system as set forth in claim 4, wherein the passage includes:
a plurality of interconnected bores extending back and forth through the block between the inlet and the outlet.

7. The system as set forth in claim 1, further including:
a microbe trapping filter between the duct and the defined region.

8. The system as set forth in claim 1, further including:
a heater and a dehumidifier connected with the duct upstream from the injection zone.

9. The system as set forth in claim 8, wherein the duct includes:
an inlet upstream of the heater and the dryer connected with the defined region such that the carrier gas is circulated from the duct inlet, through the heater and dryer, through the injection zone, and through a duct outlet into the defined region.

10. The system as set forth in claim 9 further including:
microbe trapping filters disposed adjacent the duct inlet and the duct outlet.

11. The system as set forth in claim 9 wherein the antimicrobial compound includes hydrogen peroxide and further including:
a hydrogen peroxide destroyer for decomposing hydrogen peroxide vapor into water vapor and oxygen, the destroyer being disposed upstream from the dryer.

12. The system as set forth in claim 1, further including:
a source of carrier gas connected with the flash vaporizer inlet for creating a positive pressure differential from the flash vaporizer to the absorption zone.

13. The system as set forth in claim 1 further including:
at least one additional flash vaporizer and means for introducing liquid connected with the duct.

14. The system as set forth in claim 1, further including:
at least a second duct; and,
at least a second flash vaporizer and means for introducing liquid connected with the second duct.

15. The system as set forth in claim 1 further including:
a first plurality of monitors connected with the duct upstream of the injection zone;
a second plurality of monitors disposed in the defined region;
a controller connected to the monitors for controlling the means for introducing liquid in accordance with monitored conditions in the duct and in the defined area.

16. The system as set forth in claim 1, further including:
fans disposed in the defined region for circulating vapor into partially occluded subregions.

17. The system as set forth in claim 1, wherein the means for introducing includes a metering pump.

18. The system as set forth in claim 1, further including a temporary enclosure for enclosing at least a portion of a building, the defined region including the temporary enclosure and building, the first duct being fluidly connected with the temporary enclosure.

19. A method of decontaminating a defined region, the method comprising:
pumping a carrier gas through a duct to the defined region;
injecting a mixture of an antimicrobial vapor and a carrier gas into the duct at a mixing zone upstream of the defined region.

20. The method as set forth in claim 19, wherein the defined region is contaminated with at least one of microbial contaminants and chemical contaminants and the hydrogen peroxide vapor reduces the activity of the at least one microbial or chemical contaminant.

21. The method as set forth in claim 20, wherein the contaminant is a microbial contaminant selected from the group consisting of viruses, bacteria, molds, and fungi.

22. The method as set forth in claim 21, wherein the microbial contaminant is selected from the group consisting of Stachybotrys chartarum, Aspergillus niger, Chaetomium globosum, Clostridium botulinium, Trichophyton mentagrophytes, Yersina pestis, Bacillus anthracis, Francisella tularensis, smallpox, Ebola virus, Vibrio cholerae, Fusarium, Myrotecium coccidioidomycosis, combinations thereof, and toxic products thereof.

23. The method as set forth in claim 20, wherein the microbial contaminant includes a mycotoxin and the method includes rendering the mycotoxin non-harmful to humans.

24. The method as set forth in claim 19, wherein carrier gas flow through the duct is at the rate of at least 20 cubic meters per minute and the defined area is an enclosure of at least 10,000 cubic meters.

25. The method as set forth in claim 19, wherein the antimicrobial vapor includes hydrogen peroxide and further including:
heating a block which has an internal passage to a temperature sufficient to vaporize the hydrogen peroxide but which temperature is lower than a temperature which disassociates hydrogen peroxide;
passing hydrogen peroxide into the passage through the block to vaporize the hydrogen peroxide;
passing the hydrogen peroxide vapor from the passage into the mixing zone;
mixing the hydrogen peroxide vapor into the carrier gas flow.

26. The method as set forth in claim 2, further including:
blowing carrier gas through the passage with the hydrogen peroxide to create a positive pressure differential between the passage and the duct.

27. The method as set forth in claim 25, further including heating and drying the carrier gas in the duct upstream of the mixing zone.

28. The method as set forth in claim 19, further including:
pulling carrier gas with antimicrobial vapor from the enclosed area through a microbe-trapping filter;
drying and heating the carrier gas and passing the dried, heated carrier gas to the duct upstream of the mixing zone.

29. The method as set forth in claim 28, further including anti-microbially filtering carrier gas between the duct and the defined area.

30. The method as set forth in claim 19, wherein the defined region is a large room and the duct includes existing HVAC duct work.

31. The method as set forth in claim 30, further including:
supplying carrier gas through a plurality of ducts into the room;
injecting hydrogen peroxide vapor into the carrier gas in each of the ducts.

32. The method as set forth in claim 19, wherein the method further includes:
surrounding a building with a temporary enclosure, the defined region including the building and the temporary enclosure; and
fluidly connecting the duct with at least one of the building and the enclosure.

33. The method as set forth in claim 32, wherein the decontaminating includes destroying at least one of fungi, bacteria, and viruses in the building.

34. The method as set forth in claim 33, wherein the method includes destroying fungi, the fungi being selected from the group consisting of Stachybotrys chartarum, Aspergillus niger, Chaetomium globosum, Trichophyton mentagrophytes, and combinations thereof.

35. The method as set forth in claim 19, further including:
directing antimicrobial vapor in the defined region against at least one surface to be decontaminated.

36. The method as set forth in claim 19, further including:
monitoring concentration of the antimicrobial compound in the vapor in the room and carrier gas conditions in the duct upstream of the injection zone, and
controlling a rate at which the vapor is supplied to the duct in accordance therewith.

37. The method as set forth in claim 19, further including:
monitoring concentration of the antimicrobial compound in the vapor in the defined area until the concentration reaches a preselected level; and
holding the vapor in the defined area without further addition of vapor for a period of time.

38. The method as set forth in claim 19, further including:
heating a block above a vaporization temperature of a peroxy compound;
metering the peroxy compound in liquid form into an internal bore in the block to vaporize the peroxy compound.

39. The method as set forth in claim 38, further including:
entraining the liquid peroxy compound into a controlled air flow upstream from the block.

40. The method as set forth in claim 39, wherein the internal bore turns and further including:
propelling peroxy compound droplets into bore surfaces at turns in the internal bore.

41. A method of decontaminating an enclosure comprising:
providing a first carrier gas stream and a second carrier gas stream, the first stream having a lower flow rate than the second stream:
introducing the first stream to a passage, the passage having at least one bend;
introducing a flow of an aqueous solution of a peroxy compound into the passage upstream of the bend, the peroxy compound mixing with the first stream, walls of the passage being heated to vaporize the aqueous solution;
mixing the vaporized aqueous solution and first carrier gas stream with the second carrier gas stream in a mixing zone downstream of the passage and transporting the mixed vaporized aqueous solution and first and second carrier gas streams to the enclosure.

42. The method of claim 41, wherein the flow rate of the first stream of carrier gas is less half of the flow rate of the second stream of carrier gas.

43. The method as set forth in claim 42, wherein the flow rate of the first stream of carrier gas is less than 10% of the flow rate of the second stream of carrier gas.

44. A method of destroying fungi in at least a portion of a building comprising:
connecting a duct with the at least a portion of the building;
flowing a carrier gas through the duct to the portion of the building; and
introducing a stream comprising hydrogen peroxide vapor into the flowing carrier gas in the duct, the hydrogen peroxide vapor being carried by the carrier gas into the portion of the building to destroy the fungi.

45. The method as set forth in claim 44, further including:
tenting the building prior to introducing the stream comprising hydrogen peroxide vapor into the flowing carrier gas in the duct.

46. The method as set forth in claim 44, wherein the building includes at least one of a crop storage building and a bathing facility.
